(19) United States
(12) Patent Application Publication
(10) Pub. No.: US 2021/0355376 A1
(43) Pub. Date: Nov. 18, 2021

(54) NEAR-INFRARED THERMAL ACTIVATED DELAYED FLUORESCENT MATERIAL, PREPARATION METHOD THEREOF, AND DISPLAY DEVICE

(71) Applicant: WUHAN CHINA STAR OPTOELECTRONICS SEMICONDUCTOR DISPLAY TECHNOLOGY CO., LTD., Wuhan (CN)

(72) Inventor: Jiajia LUO, Wuhan (CN)

(73) Assignee: WUHAN CHINA STAR OPTOELECTRONICS SEMICONDUCTOR DISPLAY TECHNOLOGY CO., LTD., Wuhan (CN)

(21) Appl. No.: 16/463,143

(22) PCT Filed: Dec. 29, 2018
(86) PCT No.: PCT/CN2018/125218

§ 371 Date: May 22, 2019

(30) Foreign Application Priority Data
Oct. 22, 2018 (CN) 201811228227.6

(51) Int. Cl. C09K 11/06; H01L 51/00; C07D 487/04; C07D 413/14

(52) U.S. Cl. CPC C09K 11/06; H01L 51/0071; H01L 51/0072; H01L 51/5024; C07D 413/14; C09K 2211/1018; C09K 2211/1007; C07D 487/04

(57) ABSTRACT

A near-infrared thermal activated delayed fluorescent material, a preparation method thereof and a display device are provided. The molecular structure of the near-infrared thermal activated delayed fluorescent material is a D-A-D structure or a D-A structure formed by reacting an electron donor (D) with an electron acceptor (A). The electron acceptor (A) is a planar electron acceptor (A) having a triplet energy level ranging from 1.30 to 1.80. The preparation method includes the steps of synthesizing a target compound, extracting a target compound, and purifying a target compound. The process is simple, the purification is easy, and the yield is high. The display device has a light emitting layer including the near-infrared thermally activated delayed fluorescent material, which has higher fluorescence efficiency and better stability, thereby improving the luminous efficiency and the service life of the display device.

### FIELD OF INVENTION

The invention relates to the technical field of organic electroluminescent materials, and in particular to a near-infrared thermal activated delayed fluorescent material, a preparation method thereof and a display device.

### BACKGROUND OF INVENTION

Organic electroluminescence is a luminescence phenomenon that is realized by excitation of organic material under an applied electric field and current.

In 1963, Pope et al. of New York University first observed organic electroluminescence phenomenon by using single crystal anthracene as a light-emitting layer at a driving voltage of 100 V. However, due to defects such as low luminous efficiency and high driving voltage, it did not cause widespread concern. Until 1987, C. W. Tang et al. of the Kodak Company in the United States adopted vacuum evaporation technology to obtain a novel multilayer organic light emitting diode (OLED) structure prepared by using aromatic diamine as a hole transport layer and 8-hydroxyquinoline aluminum (Alq3) as an electron transport layer and a light-emitting layer. The novel multilayer OLED structure's higher brightness, higher external quantum efficiency, and lower driving voltage inspired researchers' great interest in OLED research and provided a possibility to commercialize it. In 2012, Adachi et al. of Kyushu University in Japan reported an OLED device with a delayed fluorescent phenomenon; the OLED device has an external quantum efficiency of more than 5% and an internal quantum efficiency of more than 25%; it utilizes triplet exciton at room temperature to form singlet exciton after absorbing thermal energy to realize fluorescence emission and is therefore also referred to as thermally activated delayed fluorescence. The realization of thermally activated delayed fluorescent technology has greatly broadened the thinking of OLED research, and OLED research has entered a booming era.

For thermally activated delayed fluorescent materials, to have a fast reverse intersystem crossing constant (kRISC) and a high photoluminescence quantum yield (PLQY) is an essential requirement for preparing high-efficiency OLEDs. However, a thermally activated delayed fluorescent material meeting the above requirements such as heavy metal iridium (Ir), is still quite scarce. In the field of near-infrared light, where there is no breakthrough in the development of phosphorescent heavy metal materials, the development for the thermally activated delayed fluorescent materials is even little.

### TECHNICAL PROBLEM

An object of the present invention is to provide a near-infrared thermally activated delayed fluorescent material, a preparation method thereof, and a display device, to solve the problems of lack of thermally activated delayed fluorescent materials with excellent thermal activation delayed fluorescence characteristics and shortage of thermally activated delayed fluorescent materials in the field of near-infrared, the problems of low fluorescence efficiency and short service life in the conventional display device are also being solved.

### TECHNICAL SOLUTION

To achieve the above object, the present invention provides a near-infrared thermally activated delayed fluorescent material, the molecular structure of the near-infrared thermally activated delayed fluorescent material is a D-A-D structure or a D-A structure formed by reacting an electron donor (D) with an electron acceptor (A). The electron acceptor (A) is a planar electron acceptor having a triplet energy level ranging from 1.30 to 1.80.

Further, the compound having the electron acceptor (A) is 2,5-bis (4-bromophenyl) imidazo [4,5-d] imidazole.

Further, the compound having the electron donor (D) is at least one of 9,9-dimethylacridine, phenoxazine, and phenothiazine.

Further, the molecular structure of the near-infrared thermally activated delayed fluorescent material is one of the following molecular structures:

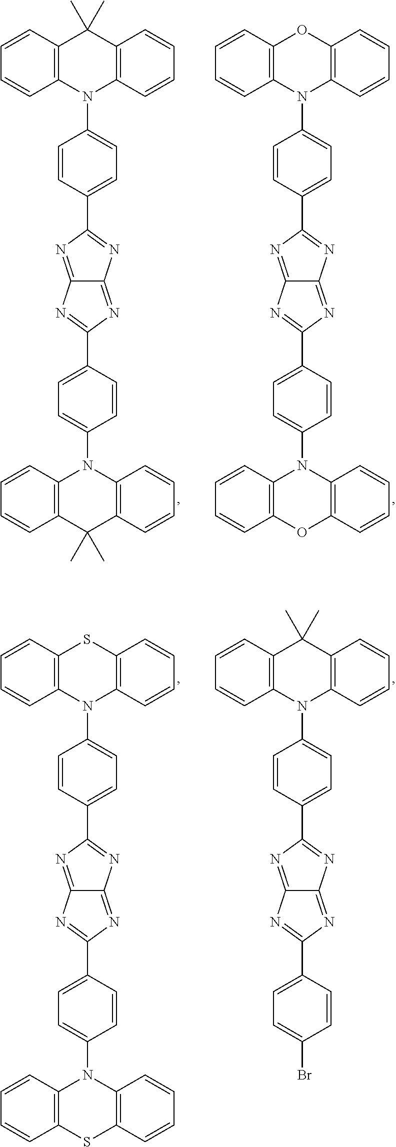

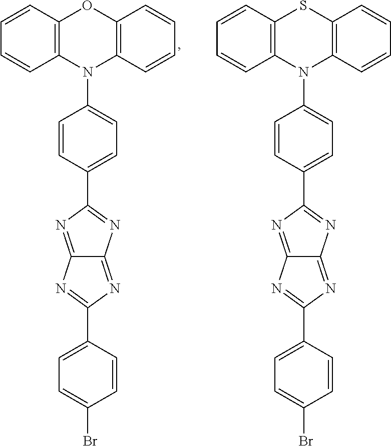

The invention further provides a method for preparing the near-infrared thermally activated delayed fluorescent material, including the following steps:

Synthesizing a target compound: Placing an electron acceptor (A), an electron donor (D) and a catalyst in a reaction vessel to obtain a reaction solution, and sufficiently reacting the reaction solution at a temperature of 100° C. to 120° C. to obtain a mixed solution, the mixed solution has a target compound obtained by the reaction;

Extracting the target compound:
Cooling the mixed solution to room temperature, extracting the target compound from the mixed solution;

Purification treatment of the target compound:
Subjecting the target compound to a purification treatment to obtain the near-infrared thermally activated delayed fluorescent material.

Further, the catalyst in the step for obtaining the reaction solution is palladium acetate, tri-tert-butyl phosphine tetrafluoroborate, sodium tert-butoxide, and toluene. The molar ratio of the electron acceptor (A), the electron donor (D), the palladium acetate, the tri-tert-butyl phosphine tetrafluoroborate, and the sodium tert-butoxide is 20:20-50:1-2:2-5:20-50.

Further, in the step of synthesizing a target compound, the electron acceptor (A), the electron donor (D), the palladium acetate and the tri-tert-butyl phosphine tetrafluoroborate are placed together in the reaction vessel, then the reaction vessel is placed in an argon atmosphere, and the sodium tert-butoxide and the dehydrated and deoxygenated toluene are added to the reaction vessel to obtain the reaction solution.

Further, the step of extracting the target compound includes: pouring the reaction solution into an ice and water mixture, adding dichloromethane thereto for extraction and combing the organic-phased extracts after multiple extractions to obtain the target compound.

Further, the step of purifying the target compound includes: performing the initial purification of the target compound by silica gel column chromatography using a developing solvent to obtain an initial purified product; purifying again the initial purified product by a re-crystallization method to obtain the near-infrared thermally activated delayed fluorescent material, wherein the developing solvent used in the silica gel column chromatography method is dichloromethane and n-hexane, and the volume ratio of the dichloromethane to the n-hexane is 1:1.

The invention further provides a display device including: a substrate, a transparent conductive layer, a hole transport layer, a light emitting layer, an electron transport layer and a cathode layer. The transparent conductive layer is disposed on the substrate; the hole transport layer is disposed on the transparent conductive layer; the light emitting layer is disposed on the hole transport layer, and the light emitting layer includes the near-infrared thermally activated delayed fluorescent material. The electron transport layer is disposed on the light emitting layer; the cathode layer is disposed on the electron transport layer.

### BENEFICIAL EFFECT

The near-infrared thermally activated delayed fluorescent material provided by the present invention adopts a D-A-D structure in which an electron donor (D) and an electron acceptor (A) are combined, it has a fast reverse intersystem crossing constant (ranging from 1*10⁴/s to 1*10⁷/s), as well as high photoluminescence quantum yield.

The electron acceptor (A) in the present invention is a planar electron acceptor (A) having an ultra-low triplet energy level, it combines an electron donor (D) having a strong electron donating ability, it adjusts the torsion angle and charge transfer characteristics between the electron donor (D) and the electron acceptor (A) to achieve the purpose of reducing the lowest single triplet level difference of the target molecule and near-infrared light emission, thereby, the target molecule has excellent luminescent performance.

Another object of the present invention is to provide a method for preparing a near-infrared thermally activated delayed fluorescent material, the process is simple, the purification is easy, and the yield is high.

Another object of the present invention is to provide a display device, in which the light emitting layer includes the near-infrared thermally activated delayed fluorescent material, so that the light emitting layer has higher fluorescence efficiency and better stability, thereby, the luminous efficiency and the service life of the display device are also improved.

### DESCRIPTION OF DRAWINGS

In order to more clearly illustrate the technical solutions in the embodiments of the present invention, the following figures described in the embodiments will be briefly introduced. It is obvious that the drawings described below are merely some embodiments of the present invention, other drawings can also be obtained by the person ordinary skilled in the field based on these drawings without doing any creative activity.

FIG. 1 is a photoluminescence spectrum of a near-infrared thermally activated delayed fluorescent material in a toluene solution according to an embodiment of the present invention;

FIG. 2 is a flow chart of preparing a near-infrared thermally activated delayed fluorescent material according to an embodiment of the present invention;

FIG. 3 is a structural cross-sectional view of a display device using the near-infrared thermally activated delayed fluorescent material in accordance with the embodiment of the present invention.

The reference numerals of the components in the figures are as follows: display device 100; substrate 101; transparent conductive layer 102; hole transport layer 103; light emitting layer 104; electron transport layer 105; cathode layer 106.

### DETAILED DESCRIPTION OF PREFERRED EMBODIMENTS

The description of the exemplary embodiments is only for the purpose of understanding the invention. It is to be understood that the present invention is not limited to the disclosed exemplary embodiments. It is obvious to those skilled in the art that the exemplary embodiments may be modified without departing from the scope and spirit of the present invention.

### Embodiment 1

The embodiment is a preferred embodiment. The molecular structure of the near-infrared thermally activated delayed fluorescent material provided in the embodiment is a DAD structure formed by the reaction of an electron donor (D) and an electron acceptor (A). The electron acceptor (A) is a planar electron acceptor (A) having a triplet energy level ranging from 1.30 to 1.80, and the electron donor (D) has a strong electron donating ability.

The compound having an electron acceptor (A) in the embodiment is 2,5-bis (4-bromophenyl) imidazo [4,5-d] imidazole, and the compound having an electron donor (D) is 9,9-dimethyl acridine.

The preparation process is shown in FIG. 2, and the specific preparation steps are as follows:

Synthesizing a target compound:
In the 100 ml two-necked flask, 2,5-bis (4-bromophenyl) imidazo [4,5-d] imidazole (2.12 g, 5 mmol), 9,9-dimethyl acridine (2.5 g, 12 mmol), palladium acetate (90 mg, 0.4 mmol) and tri-tert-butyl phosphine tetrafluoroborate are added, then, placing the reaction vessel in a tank filled with argon gas, and following the sodium tert-butoxide (1.16 g, 12 mmol) and 40 ml of dehydrated and deoxygenated toluene are added to the reaction vessel, the reaction solution is sufficiently reacted at a temperature of 110° C. to obtain a mixed solution, the mixed solution has a target compound formed by the reaction.

Extracting the target compound:
After the mixed solution is cooled to room temperature, it is poured into an ice and water mixture of 50 ml, extracted three times with dichloromethane, and the organic-phased extracts obtained by the three times extractions are combined to obtain a target compound.

Purification treatment of the target compound:
Performing an initial purification of the target compound by silica gel column chromatography using dichloromethane and n-hexane (volume ratio is 1:1) as a developing solvent to obtain a dark red powder of 2.1 g, yield 63%. The dark red powder is subjected to a secondary purification by a re-crystallization method, and finally, the near-infrared thermally activated delayed fluorescent material is obtained.

¹H NMR (300 MHz, CD₂Cl₂, δ):7.77(d, J=6.6 Hz,4H),7.28(d, J=6.0 Hz,4H),7.19-7.14(m,12H),7.01-7.96(m,4H),1.69(s,12H).MS(EI)m/z: [M]⁺ calcd for C₄₆H₃₆N₆,672.3; found,657.2 [M-CH3]⁺.Anal. Calcd for C₄₆H₃₆N₆:C 82.12,H 5.39,N 12.49;found:C 82.09,H 5.23,N 12.28.

The preparation process is shown in formula 1:

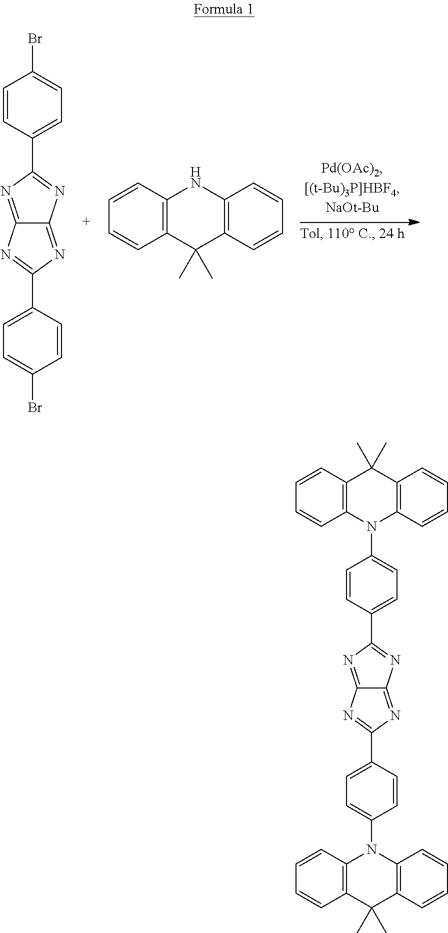 [SMILES: Brc1ccc(C2=NC3=NC(c4ccc(Br)cc4)=NC3=N2)cc1 ; CC1(C)c2ccccc2Nc2ccccc21 ; CC1(C)c2ccccc2N(c2ccc(C3=NC4=NC(c5ccc(N6c7ccccc7C(C)(C)c7ccccc76)cc5)=NC4=N3)cc2)c2ccccc21]

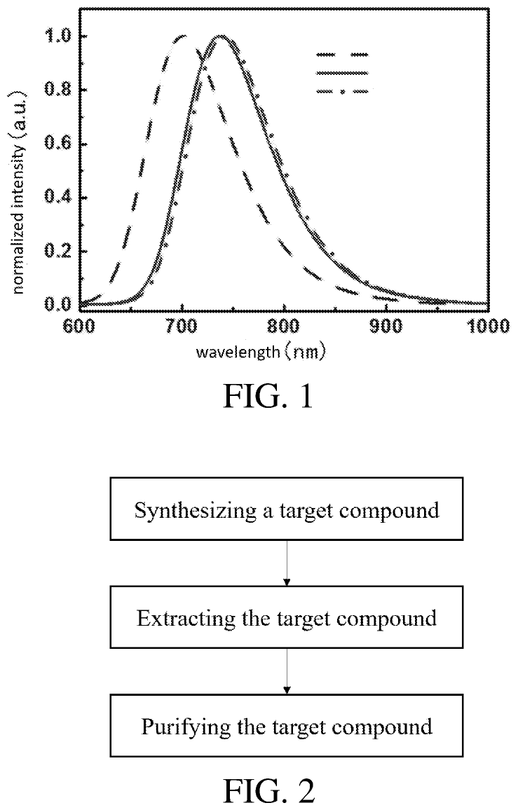

The prepared near-infrared thermally activated delayed fluorescent material at room temperature in toluene solution has a photoluminescence spectrum as shown in FIG. 1. Its photoluminescence peak (PL Peak), lowest singlet state (S₁), lowest triplet energy level (T₁), and electrochemical energy level are shown in Table 1.

TABLE 1

| PL Peak(nm) | S₁(eV) | T₁(eV) | E_{ST} (eV) | HOMO (eV) | LUMO (eV) |
|---|---|---|---|---|---|
| 703 | 1.77 | 1.59 | 0.08 | −5.11 | −3.86 |

In the embodiment, the near-infrared thermally activated delayed fluorescent material combines a planar electron acceptor (A) having an ultra-low triplet energy level and an electron donor (D) having a stronger electron donating ability, it adjusts torsion angle and charge transfer characteristics between the electron donor (D) and the electron acceptor (A) to achieve a purpose of reducing the lowest single triplet level difference of the target molecule and near-infrared light emission, thereby, the target molecule has a fast reverse intersystem crossing constant (ranging from 1*10⁴/s to 1*10⁷/s), as well as high photoluminescence quantum yield. The preparation process provided in the present embodiment is simple, the purification is easy, and the yield is high.

In the embodiment, a near-infrared thermally activated delayed fluorescent material having a molecular structure of D-A structure can also be formed, and the preparation process thereof is shown in formula 2:

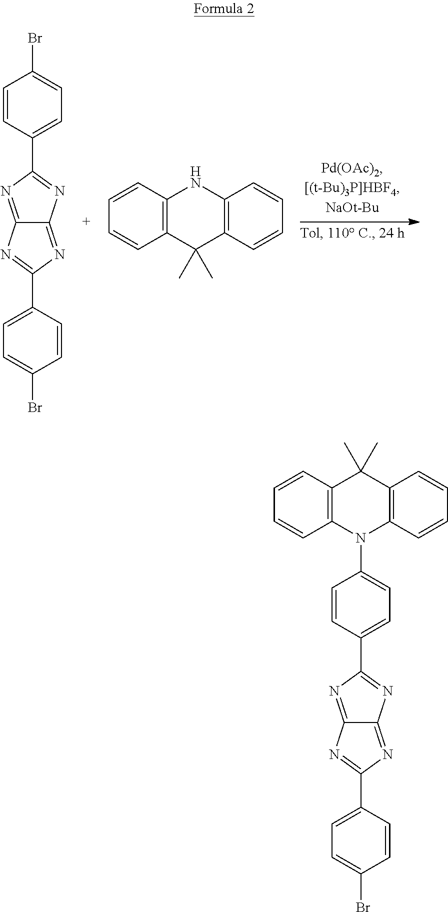 [SMILES: Brc1ccc(C2=NC3=NC(c4ccc(Br)cc4)=NC3=N2)cc1 ; CC1(C)c2ccccc2Nc2ccccc21 ; CC1(C)c2ccccc2N(c2ccc(C3=NC4=NC(c5ccc(Br)cc5)=NC4=N3)cc2)c2ccccc21]

### Embodiment 2

The near-infrared thermally activated delayed fluorescent material provided by the present invention adopts a D-A-D structure in which an electron donor (D) and an electron acceptor (A) are combined, the electron acceptor (A) is a planar electron acceptor having a triplet energy level ranging from 1.30 to 1.80, and the electron donor (D) having a stronger electron donating ability.

The compound having an electron acceptor (A) in the embodiment is 2,5-bis (4-bromophenyl) imidazo [4,5-d] imidazole, and the compound having an electron donor (D) is phenoxazine. The preparation process is shown in FIG. 2, and the specific preparation steps are as follows:

Synthesizing a target compound:

In the 100 ml two-necked flask, 2,5-bis (4-bromophenyl) imidazo [4,5-d] imidazole (2.12 g, 5 mmol), phenoxazine (2.2 g, 12 mmol), palladium acetate (90 mg, 0.4 mmol) and tri-tert-butyl phosphine tetrafluoroborate (0.34 g, 1.2 mmol) are added, then the reaction vessel is placed in a tank filled with argon gas, and following the sodium tert-butoxide (1.16 g, 12 mmol) and 40 ml of dehydrated and deoxygenated toluene are added to the reaction vessel, the reaction solution is sufficiently reacted at a temperature of 110° C. to obtain a mixed solution having a target compound formed by the reaction.

Extracting the target compound:
After the mixed solution is cooled to room temperature, it is poured into an ice and water mixture of 50 ml, extracted three times with dichloromethane, and the organic-phased extracts obtained by three times extractions are combined to obtain a target compound.

Purification treatment of the target compound:

Performing an initial purification of the target compound by silica gel column chromatography using dichloromethane and n-hexane (volume ratio is 1:1) as a developing solvent to obtain a dark red powder of 1.86 g, yield 48%. The dark red powder is subjected to a secondary purification by a re-crystallization method, and finally, the near-infrared thermally activated delayed fluorescent material of 1.03 g is obtained.

¹H NMR (300 MHz,CD₂Cl₂,δ):7.77(d, J=6.9 Hz,4H),7.28(d, J=6.0 Hz,4H),7.19 (d, J=6.3 Hz,4H),7.03-6.95(m,12H).MS (EI)m/z:[M]⁺ calcd for C₄₀H₂₄N_{6O2},620.20; found,620.18.Anal.Calcd for C₄₀H₂₄N_{6O2}:C 77.41,H 3.90,N 13.54;found:C 77.25,H 3.77,N 13.41.

The preparation process is shown in formula 3:

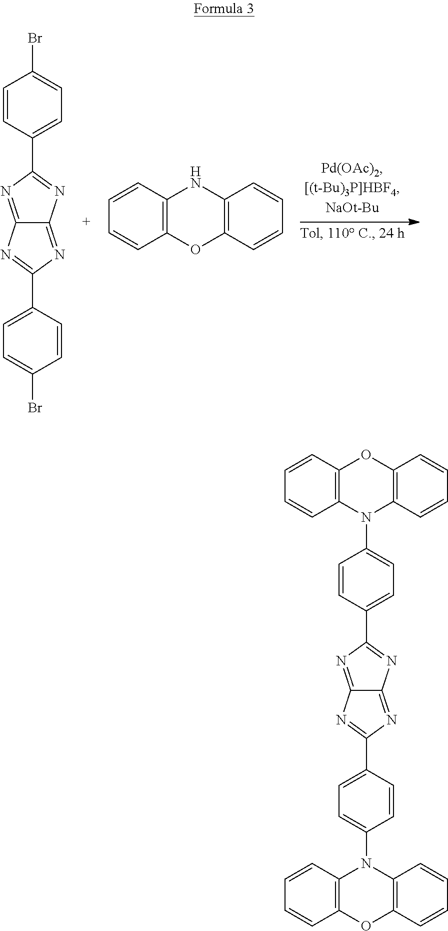 [SMILES: Brc1ccc(C2=NC3=NC(c4ccc(Br)cc4)=NC3=N2)cc1 ; c1ccc2c(c1)Nc1ccccc1O2 ; c1ccc2c(c1)Oc1ccccc1N2c1ccc(C2=NC3=NC(c4ccc(N5c6ccccc6Oc6ccccc65)cc4)=NC3=N2)cc1]

The prepared near-infrared thermally activated delayed fluorescent material at room temperature in toluene solution has a photoluminescence spectrum as shown in FIG. 2. Its photoluminescence peak (PL Peak), lowest singlet state (S1), lowest triplet energy level (T1), and electrochemical energy level are shown in Table 2.

TABLE 2

| PL Peak(nm) | S₁(eV) | T₁(eV) | E_{ST} (eV) | HOMO (eV) | LUMO (eV) |
|---|---|---|---|---|---|
| 739 | 1.68 | 1.61 | 0.07 | −4.97 | −3.71 |

In the embodiment, the near-infrared thermally activated delayed fluorescent material combines a planar electron acceptor (A) having an ultra-low triplet energy level and an electron donor (D) having a stronger electron donating ability, it adjusts torsion angle and charge transfer characteristics between the electron donor (D) and the electron acceptor (A) to achieve the purpose of reducing the lowest single triplet level difference of the target molecule and near-infrared light emission, thereby, the target molecule has a fast reverse intersystem crossing constant (ranging from 1*10⁴/s to 1*10⁷/s), as well as high photoluminescence quantum yield. The preparation process provided in the present embodiment is simple, the purification is easy, and the yield is high.

In the embodiment, a near-infrared thermally activated delayed fluorescent material having a molecular structure of D-A structure can also be formed, and the preparation process thereof is shown in formula 4:

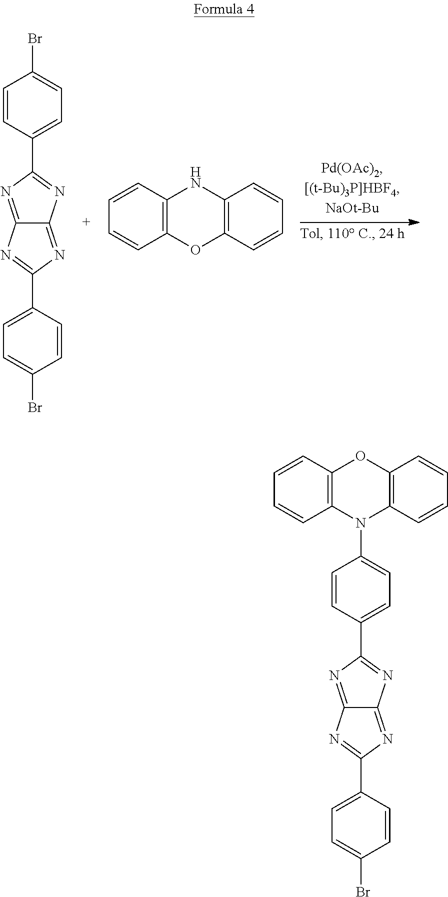 [SMILES: Brc1ccc(C2=NC3=NC(c4ccc(Br)cc4)=NC3=N2)cc1 ; c1ccc2c(c1)Nc1ccccc1O2 ; Brc1ccc(C2=NC3=NC(c4ccc(N5c6ccccc6Oc6ccccc65)cc4)=NC3=N2)cc1]

### Embodiment 3

The near-infrared thermally activated delayed fluorescent material provided by the present invention adopts a D-A-D structure in which an electron donor (D) and an electron acceptor (A) are combined, the electron acceptor (A) is a planar electron acceptor having a triplet energy level ranging from 1.30 to 1.80, and the electron donor (D) having a stronger electron donating ability.

The compound having an electron acceptor (A) in the embodiment is 2,5-bis (4-bromophenyl) imidazo [4,5-d] imidazole, and the compound having an electron donor (D) is phenoxazine.

The preparation process is shown in FIG. 2, and the specific preparation steps are as follows:

Synthesizing a target compound:

In the 100 ml two-necked flask, 2,5-bis (4-bromophenyl) imidazo [4,5-d] imidazole (2.12 g, 5 mmol), phenoxazine (2.2 g, 12 mmol), palladium acetate (90 mg, 0.4 mmol) and tri-tert-butyl phosphine tetrafluoroborate (0.34 g, 1.2 mmol) are added, then the reaction vessel is placed in a tank filled with argon gas, and following sodium tert-butoxide (1.16 g, 12 mmol) and 40 ml of dehydrated and deoxygenated toluene are added to the reaction vessel, the reaction solution is sufficiently reacted at a temperature of 110° C. to obtain a mixed solution having a target compound formed by the reaction.

Extracting the target compound:
After the mixed solution is cooled to room temperature, it is poured into an ice and water mixture of 50 ml, extracted three times with dichloromethane, and the organic-phased extracts obtained by three times extractions are combined to obtain a target compound.

Purification treatment of the target compound:
Performing an initial purification of the target compound by silica gel column chromatography using dichloromethane and n-hexane (volume ratio is 1:1) as a developing solvent to obtain a dark red powder of 1.34 g, yield 41%. The dark red powder is subjected to a secondary purification by a re-crystallization method, and finally, the near-infrared thermally activated delayed fluorescent material of 0.98 g is obtained.

¹H NMR (300 MHz, CD₂Cl₂, δ):7.79(d, J=6.6 Hz,4H),7.26(d, J=6. Hz,4H),7.24-7.19(m,12H),7.03-6.96(m,4H).MS (EI)m/z:[M]⁺ calcd for C₄₀H₂₄N₆S₂,652.15; found,652.12.Anal.Calcd for C₄₀H₂₄N_{6O2}: C 73.60,H 3.71,N 12.87;found:C 73.45,H 3.57,N 12.69.

The preparation process is shown in formula 5:

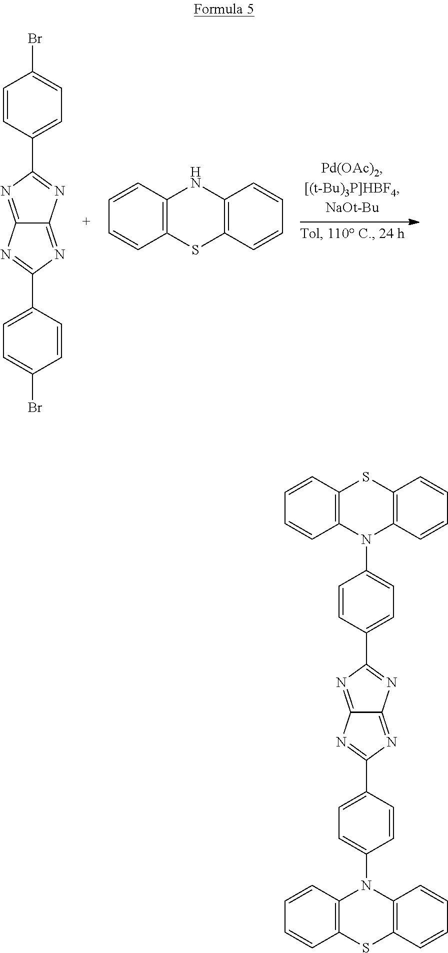 [SMILES: Brc1ccc(C2=NC3=NC(c4ccc(Br)cc4)=NC3=N2)cc1 ; c1ccc2c(c1)Nc1ccccc1S2 ; c1ccc2c(c1)Sc1ccccc1N2c1ccc(C2=NC3=NC(c4ccc(N5c6ccccc6Sc6ccccc65)cc4)=NC3=N2)cc1]

The prepared near-infrared thermally activated delayed fluorescent material at room temperature in toluene solution has a photoluminescence spectrum as shown in FIG. 1.

Its photoluminescence peak (PL Peak), lowest singlet state (S1), lowest triplet energy level (T1), and electrochemical energy level are shown in Table 3.

TABLE 3

| PL Peak(nm) | S₁(eV) | T₁(eV) | E_{ST} (eV) | HOMO (eV) | LUMO (eV) |
|---|---|---|---|---|---|
| 743 | 1.67 | 1.51 | 0.16 | −5.19 | −3.97 |

In the embodiment, the near-infrared thermally activated delayed fluorescent material combines a planar electron acceptor (A) having an ultra-low triplet energy level and an electron donor (D) having a stronger electron donating ability, it adjusts torsion angle and charge transfer characteristics between the electron donor (D) and the electron acceptor (A) to achieve the purpose of reducing the lowest single triplet level difference of the target molecule and near-infrared light emission, thereby, the target molecule has a fast reverse intersystem crossing constant (ranging from 1*10⁴/s to 1*10⁷/s), as well as high photoluminescence quantum yield.

The preparation process provided in the present embodiment is simple, the purification is easy, and the yield is high.

In the embodiment, a near-infrared thermally activated delayed fluorescent material having a molecular structure of D-A structure can also be formed, and the preparation process thereof is shown in formula 6:

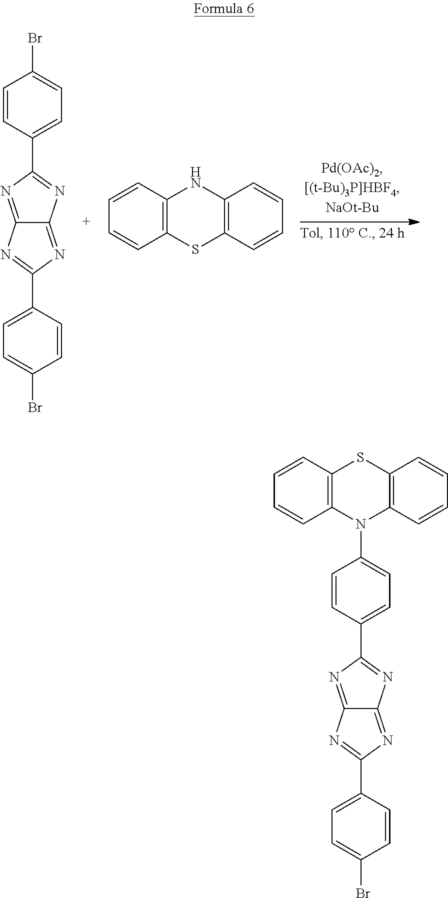 [SMILES: Brc1ccc(C2=NC3=NC(c4ccc(Br)cc4)=NC3=N2)cc1 ; c1ccc2c(c1)Nc1ccccc1S2 ; Brc1ccc(C2=NC3=NC(c4ccc(N5c6ccccc6Sc6ccccc65)cc4)=NC3=N2)cc1]

### Applied Embodiment

The embodiment is an application of the near-infrared thermally-activated delayed fluorescent material prepared in embodiments 1 to 3, and it is applied to a display device. In the embodiment, an OLED display device is taken as an example to describe the display device in the present embodiment.

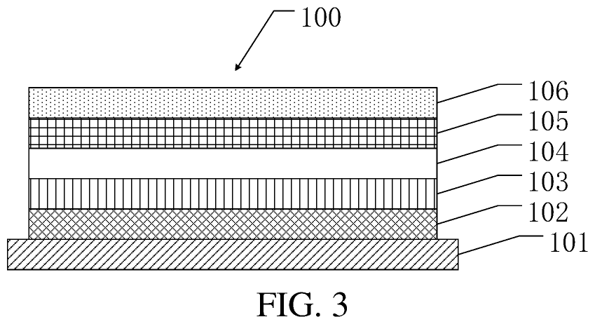

As shown in FIG. 3, the embodiment provides a display device 100, including a substrate 101, a transparent conductive layer 102, a hole transport layer 103, a light emitting layer 104, an electron transport layer 105, and a cathode layer 106.

The substrate 101 is a glass substrate for protecting the overall structure of the display device 100. The transparent conductive layer 102 is on the substrate 101, it is an indium tin oxide transparent conductive glass for transmitting current.

The hole transport layer 103 is 4-ethylenedioxythiophene:polystyrene sulfonate (PEOT:PSS) which is a P-electrode layer of the display device 100.

The electron transport layer 105 is 1,3,5-tris (3-(3-pyridyl) phenyl) benzene (benzene Tm3PyPB) which is an N-electrode layer of the display device 100.

The hole transport layer 103 and the electron transport layer 105 transport electrons to the light-emitting layer 104 in accordance with the PN junction principle.

The light-emitting layer 104 includes a near-infrared thermally activated delayed fluorescent material that converts electrical energy into light energy, thereby provide a light source for the display device 100.

In order to clearly illustrate the performance of the near-infrared thermally activated delayed fluorescent material of the present invention, a final product of the materials prepared in embodiments 1 to 3, i.e. the display device 100 is subjected to a performance test, wherein in Table 4 the device lrespresents the display device 100 having the near-infrared thermally activated delayed fluorescent material prepared in accordance with embodiment 1, the device 2 represents the display 100 having the near-infrared thermally activated delayed fluorescent material prepared in accordance with embodiment 2, and the device 3 represents the display device 100 having the near-infrared thermally activated delayed fluorescent material prepared in accordance with embodiment 3.

The test mainly measured the highest brightness, the highest current efficiency, the electroluminescence peak (EL Peak) and the maximum external quantum efficiency, the data is shown in Table 4.

TABLE 4

| | | | | maximum |
|---|---|---|---|---|
| | | highest | | external |
| | highest | current | EL | quantum |
| | brightness | efficiency | Peak | efficiency |
| device | (cd/m2) | (cd/A) | (nm) | (%) |
| device 1 | 1235 | 5.8 | 698 | 4.8 |
| device 2 | 986 | 3.7 | 735 | 3.7 |
| device 3 | 523 | 2.0 | 738 | 2.2 |

The measurements of the current, the brightness and the voltage characteristics of the devices are performed by a Keithley source measurement system (Keithley 2400 Sourcemeter, Keithley 2000 Currentmeter) with a calibrated silicon photodiode, the electroluminescence spectrum is measured by the French JY Company SPEX CCD 3000 spectrometer, and all measurements are done at room temperature in the atmosphere.

As shown in Table 4, the near-infrared thermally activated delayed fluorescent material of the present invention has excellent luminescent properties, and the light emitting layer 104 produced therefrom has higher fluorescence efficiency and better stability, thereby also improving luminous efficiency and service life of the display device 100.

The description of the above exemplary embodiments is only for the purpose of understanding the invention. It is to be understood that the present invention is not limited to the disclosed exemplary embodiments. It is obvious to those skilled in the art that the above exemplary embodiments may be modified without departing from the scope and spirit of the present invention.

What is claimed is:

1. A near-infrared thermally activated delayed fluorescent material, wherein the molecular structure of the near-infrared thermally activated delayed fluorescent material is a D-A-D structure or a D-A structure formed by reacting an electron donor (D) with an electron acceptor (A), and wherein the electron acceptor (A) is a planar electron acceptor having a triplet energy level ranging from 1.30 to 1.80.

2. The near-infrared thermally activated delayed fluorescent material according to claim 1, wherein a compound having the electron acceptor (A) is 2,5-bis (4-bromophenyl) imidazo [4,5-d] imidazole.

3. The near-infrared thermally activated delayed fluorescent material according to claim 1, wherein a compound having the electron donor (D) is at least one of 9,9-dimethyl acridine, phenoxazine and phenothiazine.

4. The near-infrared thermally activated delayed fluorescent material according to claim 1, wherein a molecular structure of the near-infrared thermally activated delayed fluorescent material is one of the following molecular structures:

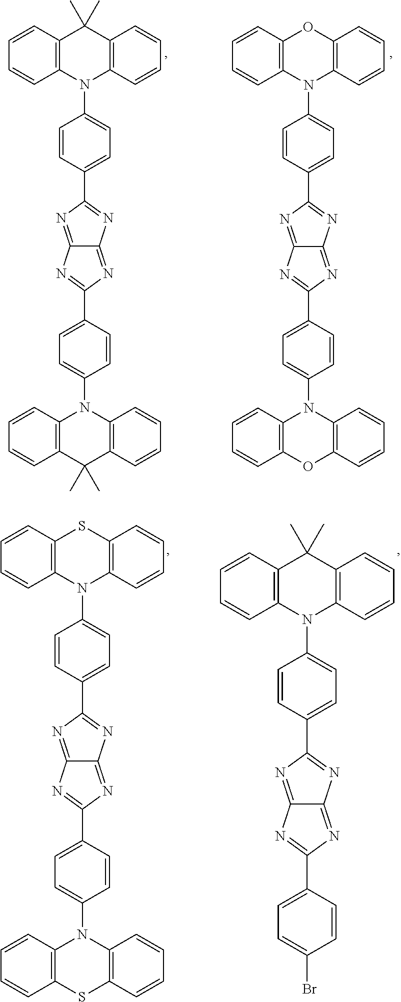

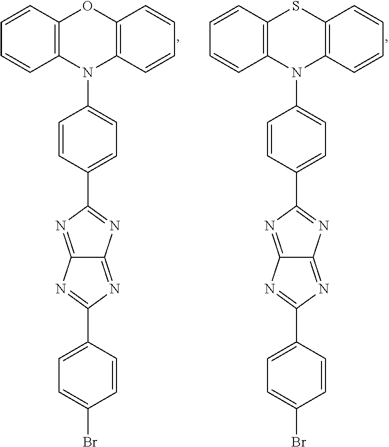

5. A method of preparing a near-infrared thermally activated delayed fluorescent material, comprising the steps of:
synthesizing a target compound comprising:
placing an electron acceptor (A), an electron donor (D) and a catalyst in a reaction vessel to obtain a reaction solution, and sufficiently reacting the reaction solution at a temperature of 100° C. to 120° C. to obtain a mixed solution, the mixed solution having the target compound obtained by the reaction;
extracting the target compound comprising:
cooling the mixed solution to room temperature, extracting the target compound from the mixed solution;
purifying of the target compound comprising:
subjecting the target compound to a purification treatment to obtain the near-infrared thermally activated delayed fluorescent material.

6. The method of preparing the near-infrared thermally activated delayed fluorescent material according to claim 5, wherein the catalyst in the step for obtaining the reaction solution is palladium acetate, tri-tert-butyl phosphine tetrafluoroborate, sodium tert-butoxide, and toluene, and the molar ratio of the electron acceptor (A), the electron donor (D), the palladium acetate, the tri-tert-butyl phosphine tetrafluoroborate, and the sodium tert-butoxide is 20:20-50:1-2:2-5:20-50.

7. The method of preparing the near-infrared thermally activated delayed fluorescent material according to claim 6, wherein in the step of synthesizing a target compound, the electron acceptor (A), the electron donor (D), the palladium acetate and the tri-tert-butyl phosphine tetrafluoroborate are placed together in the reaction vessel, then the reaction vessel is placed in an argon atmosphere, and the sodium tert-butoxide and the dehydrated and deoxygenated toluene are added to the reaction vessel to obtain the reaction solution.

8. The method of preparing the near-infrared thermally activated delayed fluorescent material according to claim 5, wherein, the step of extracting the target compound includes:
pouring the reaction solution into an ice and water mixture, adding dichloromethane thereto for extraction, and combing the organic-phased extracts after multiple extractions to obtain the target compound.

9. The method of preparing the near-infrared thermally activated delayed fluorescent material according to claim 5, wherein the step of purifying the target compound includes:
performing the initial purification of the target compound by silica gel column chromatography using a developing solvent to obtain an initial purified product;
purifying again the initial purified product by a re-crystallization method to obtain the near-infrared thermally activated delayed fluorescent material, wherein the developing solvent used in the silica gel column chromatography method is dichloromethane and n-hexane, and the volume ratio of the dichloromethane to the n-hexane is 1:1.

10. A display device comprising:
a substrate;
a transparent conductive layer disposed on the substrate;
a hole transport layer disposed on the transparent conductive layer;
a light emitting layer disposed on the hole transport layer;
an electron transport layer disposed on the light emitting layer;
a cathode layer disposed on the electron transport layer; wherein the light emitting layer comprises the near-infrared thermally activated delayed fluorescent material according to claim 1.
